(19) United States
(12) Patent Application Publication
(10) Pub. No.: US 2026/0157792 A1
(43) Pub. Date: Jun. 11, 2026

(54) APPARATUSES FOR CATHETER-BASED ELECTROPHYSIOLOGY

(71) Applicant: Biosense Webster (Israel) Ltd., Yokneam (IL)

(72) Inventors: Joseph Thomas KEYES, Sierra Madre, CA (US); Christopher Thomas BEECKLER, Brea, CA (US); Larissa ALEXANDER, Rancho Santa Margarita, CA (US)

(73) Assignee: Biosense Webster (Israel) Ltd., Yokneam (IL)

(21) Appl. No.: 18/969,596

(22) Filed: Dec. 5, 2024

(51) Int. Cl. A61B 18/14; A61B 18/00; B23K 26/362

(52) U.S. Cl. CPC A61B 18/1492; B23K 26/362; A61B 2018/00077; A61B 2018/00083; A61B 2018/00267; A61B 2018/00357; A61B 2018/00577; A61B 2018/00642; A61B 2018/00839; A61B 2018/1467

(57) ABSTRACT

The present disclosure provides a flexible circuit strip for use in a catheter assembly. The flexible circuit strip comprises a first insulating layer, a second insulating layer and a third insulating layer, each having an elongated shape and bonded together such that the second insulating layer is between the first and third insulating layers; a conductive trace layer positioned between the first and second insulating layers, the conductive trace layer including longitudinal circuit traces for carrying electrical signals; electrodes positioned along a length of an outer surface of the first insulating layer and electrically connected to the circuit traces; and a longitudinal hollow channel formed between any two of the first, second and third layers, the longitudinal hollow channel being configured to receive an elongated support element.

### FIELD

The presently disclosed subject matter generally relates to the field of medical devices and in particular to catheters for electrophysiology.

### BACKGROUND

Cardiac arrhythmias, such as atrial fibrillation (AF), occur when regions of cardiac tissue abnormally conduct electric signals to adjacent tissue. This disrupts the normal cardiac cycle and causes asynchronous rhythm. Certain procedures exist for treating arrhythmia, including surgically disrupting the origin of the signals causing the arrhythmia and disrupting the conducting pathway for such signals. By selectively ablating cardiac tissue by application of energy via a catheter, it is sometimes possible to cease or modify the propagation of unwanted electrical signals from one portion of the heart to another.

Many current ablation approaches in the art tend to utilize radiofrequency (RF) electrical energy to heat tissue. RF ablation can have certain rare drawbacks due to operator's skill, such as heightened risk of thermal cell injury which can lead to tissue charring, burning, steam pop, phrenic nerve palsy, pulmonary vein stenosis, and esophageal fistula. Cryoablation is an alternative approach to RF ablation that can reduce some thermal risks associated with RF ablation but may present tissue damage due to the very low temperature nature of such devices. Maneuvering cryoablation devices and selectively applying cryoablation, however, is generally more challenging compared to RF ablation; therefore, cryoablation is not viable in certain anatomical geometries which may be reached by electrical ablation devices.

Some ablation approaches use irreversible electroporation (IRE) to ablate cardiac tissue using nonthermal ablation methods. IRE delivers short pulses of high voltage to tissues and generates an unrecoverable permeabilization of cell membranes. Delivery of IRE energy to tissues using multi-electrode catheters was previously proposed in the patent literature. Examples of systems and devices configured for IRE ablation are disclosed in U.S. Patent Pub. No. 2021/0169550A1, 2021/0169567A1, 2021/0169568A1, 2021/0161592A1, 2021/0196372A1, 2021/0177503A1, AND 2021/0186604A1.

Regions of cardiac tissue can be mapped by a catheter to identify the abnormal electrical signals. The same or different catheter can be used to perform ablation. Some example catheters include a number of spines with electrodes positioned thereon. The electrodes are generally attached to the spines and secured in place by soldering, welding, or using an adhesive. Furthermore, multiple linear spines are generally assembled together by attaching both ends of the linear spines to a tubular shaft (e.g., a pusher tube) to form a spherical basket. Due to the small size of the spines and the electrodes, however, adhering the electrodes to the spines and then forming a spherical basket from the multiple linear spines can be a difficult task, increasing the manufacturing time and cost and the chances that the electrode fails due to an improper bond or misalignment. What is needed, therefore, are devices and methods of forming an improved basket assembly that can help to reduce the time required for manufacturing the basket assembly, alternative catheter geometries, and alternative electrode shapes and sizes in general.

U.S. Patent Application Publication No. 2010/0305420 A1 provides a flexible circuit

that includes conductive material on the top and bottom planar surfaces of a dielectric substrate. The flexible circuit can be used in various applications, including use as a sensor. A via is used to provide electrical communication between the top and bottom surface of the flexible circuit. A method of preparing a flexible circuit and a medical instrument including the flexible circuit are also provided.

U.S. Pat. No. 9,171,794 B2 provides systems and methods for the embedding of thin chips. A well region is generated in a substrate that includes a conductive material disposed on a flexible polymer. The standoff well region can be generated by pattern the conductive material, where the thin chip is embedded in the standoff well region. A cavity can be generated in the polymer layer to form a polymer well region, where the thin chip is embedded in the polymer well region.

U.S. Pat. No. 10,362,953 B2 provides a catheter with an electrode array formed by an interconnected framework. The framework may have a plurality of elements interconnected by a plurality of junctions at locations intermediate the proximal and distal ends of the electrode array assembly. The electrodes may be printed on a polymeric layer of the interconnected framework.

U.S. Pat. No. 10,297,572 B2 provides flexible interconnects, flexible integrated circuit systems and devices, and methods of making and using flexible integrated circuitry. A flexible integrated circuit system is disclosed which includes first and second discrete devices that are electrically connected by a discrete flexible interconnect. The first discrete devices include a first flexible multi-layer integrated circuit (IC) package with a first electrical connection pad on an outer surface thereof. The second discrete device includes a second flexible multi-layer integrated circuit (IC) package with a second electrical connection pad on an outer surface thereof. The discrete flexible interconnect is attached to and electrically connects the first electrical connection pad of the first discrete device to the second electrical connection pad of the second discrete device.

U.S. Pat. No. 10,681,805 B2 provides an improved flexible circuit structure, for example, by providing fault-tolerant electrical pathways for flow of electric current through the flexible circuit structure. In some embodiments, such fault tolerance is enhanced by way of a conductive mesh provided between an adjacent pair of resistive elements. Some aspects are related to improved voltage, current, or voltage and current measurement associated with various pairs of adjacent resistive elements at least when the various pairs have differing distances between them.

U.S. Pat. No. 9,159,635 B2 provides a flexible electronic structure and methods for fabricating flexible electronic structures. An example method includes applying a first layer to a substrate, creating a plurality of vias through the first layer to the substrate, and applying a second polymer layer to the first layer such that the second polymer forms anchors contacting at least a portion of the substrate. At least one electronic device layer is disposed on a portion of the second polymer layer. At least one trench is formed through the second polymer layer to expose at least a portion of the first layer. At least a portion of the first layer is removed by exposing the structure to a selective etchant to providing a flexible electronic structure that is in contact with the substrate. The electronic structure can be released from the substrate.

### SUMMARY

The present disclosure addresses the technical challenges associated with manufacturing catheters for use in catheter-based electrophysiology, particularly for mapping and ablation procedures. Electrophysiology catheters typically include an expandable end effector including flexible splines forming-in its an expanded form-a basket shape. Current methods for manufacturing such flexible splines include laminating flexible circuit strips onto elongated flexible support members, such as nitinol and use of heat shrink sleeves. These processes become increasingly complex and costly when dealing with shape-set nitinol or other materials with intricate three-dimensional geometries. In particular, traditional methods are prone to issues like improper bonding, misalignment, and the need for costly and labor-intensive steps such as laser ablation to create openings for exposing the electrodes subsequently to the use of heat shrink sleeves.

The presently disclosed subject matter proposes creating a flexible circuit strip that incorporates a longitudinal hollow channel during the fabrication process. This channel is designed to accommodate the elongated support element, such as shape-set nitinol, without the need for numerous additional assembly steps post-fabrication. By integrating the channel into the flexible circuit strip, the assembly process is simplified, reducing time and cost while improving the reliability and performance of the final product.

The longitudinal hollow channel may in particular be formed by etching away a conductive layer (e.g., copper) while preserving other conductive elements such as gold traces and electrodes. This channel is designed to receive an elongated support element, allowing for ease of assembly. The flexible circuit strip with its integrated channel can accommodate shape-set nitinol and other materials with various geometries.

### BRIEF DESCRIPTION OF THE DRAWINGS

In order to better understand the subject matter that is disclosed herein and to exemplify how it may be carried out in practice, examples will now be described, by way of non-limiting example only, with reference to the accompanying drawings, in which:

FIG. 1 illustrates a catheter-based electrophysiology mapping and ablation system, according to examples of the presently disclosed subject matter;

FIG. 2 illustrates a catheter assembly according to examples of the present disclosure;

FIG. 3 illustrates a top view of flexible circuit strips according to examples of the present disclosure;

FIG. 4 illustrates a cross section view of a flexible circuit strip according to examples of the present disclosure;

FIGS. 5A-5C illustrate views of a flexibles circuit strip according to other examples of the present disclosure;

FIG. 6 illustrates steps of a method for fabricating a flexible circuit strip according to examples of the present disclosure.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” can refer to the range of values ±20% of the recited value, e.g. “about 90%” can refer to the range of values from 71% to 99%.

As discussed herein, vasculature of a “subject” or “patient” can be vasculature of a human or any animal. It should be appreciated that an animal can be a variety of any applicable type, including, but not limited thereto, mammal, veterinarian animal, livestock animal or pet type animal, etc. As an example, the animal can be a laboratory animal specifically selected to have certain characteristics similar to a human (e.g., rat, dog, pig, monkey, or the like). It should be appreciated that the subject can be any applicable human patient, for example.

As discussed herein, “operator” can include a doctor, physician, surgeon, or any other individual or delivery instrumentation associated with delivery of a multi-electrode RF balloon catheter for the treatment of drug refractory atrial fibrillation to a subject and the term “proximal” means that an object is closer to the “operator” and “distal” means that the object is further away from the “operator”.

As used herein in the context of a circuit strip, the term ‘longitudinal’ refers to the direction along the length of the strip, extending from the proximal end to the distal end. The term ‘lateral’ refers to the direction perpendicular to the longitudinal axis, spanning the width of the strip. The term ‘thickness’ refers to the dimension perpendicular to both the longitudinal and lateral directions, indicating the depth of the strip from the top surface (i.e. top layer) to the bottom surface (bottom layer).

In the following detailed description, numerous specific details are set forth in order to provide a thorough understanding. However, it will be understood by those skilled in the art that the presently disclosed subject matter may be practiced without these specific details. In other instances, well-known methods and features have not been described in detail so as not to obscure the presently disclosed subject matter.

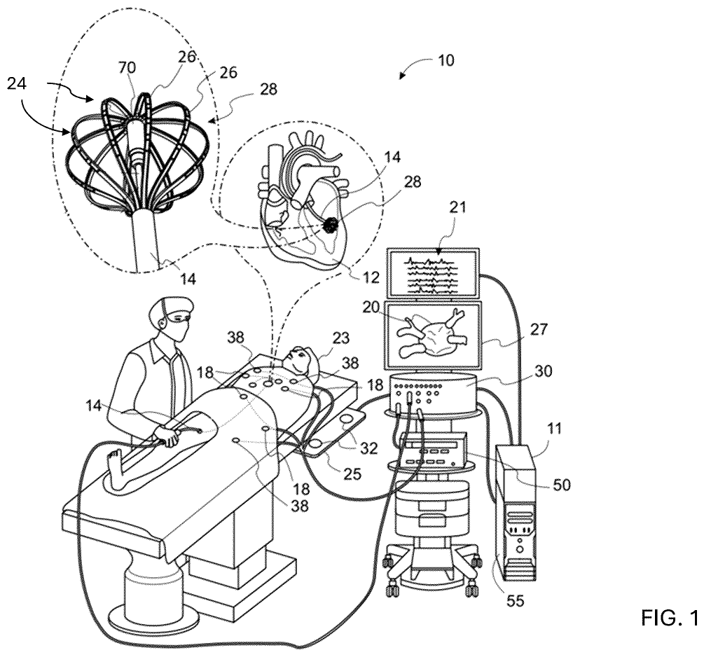

Reference is made to FIG. 1 showing an example catheter-based electrophysiology mapping and ablation system 10. System 10 includes multiple catheters, which are percutaneously inserted by a physician through the patient's vascular system into a chamber or vascular structure of a heart 12. Typically, a delivery sheath catheter is inserted into the left or right atrium near a desired location in heart 12. Thereafter, one or more catheters may be inserted into the delivery sheath catheter so as to arrive at the desired location in heart 12. The plurality of catheters may include catheters dedicated for sensing Intracardiac Electrogram (IEGM) signals, catheters dedicated for ablating and/or catheters dedicated for both sensing and ablating. An example catheter 14 that is configured for ablating tissue and/or for sensing electrical cardiac activity and/or mapping is illustrated herein.

Catheter 14 is an exemplary catheter having an end effector at its distal tip 28 that includes an expandable assembly, having one and preferably multiple electrodes 26 optionally distributed over a plurality of flexible spline elements 24. The electrodes 26 are generally configured for delivering ablation energy to tissue and/or for sensing cardiac electrical signals. Catheter 14 additionally includes one or more position sensors 70 embedded in or near distal tip 28 for tracking position and orientation of distal tip 28. Optionally and preferably, position sensor 70 is a magnetic based position sensor, for example a position sensor including three magnetic coils for sensing three-dimensional (3D) position and orientation; or a position sensor including one magnetic coil, for sensing a single direction.

Each of the magnetic based position sensors 70 may be operated together with a location pad 25 including a plurality of magnetic coils 32 configured to generate magnetic fields in a predefined working volume. Real time position of distal tip 28 of catheter 14 may be tracked based on magnetic fields generated with location pad 25 and sensed by magnetic based position sensor 70. Details of the magnetic based position sensing technology are described in U.S. Pat. Nos. 5,391,199; 5,443,489; 5,558,091; 6,172,499; 6,239,724; 6,332,089; 6,484,118; 6,618,612; 6,690,963; 6,788,967; 6,892,091.

The physician may place a distal tip 28 of catheter 14 in contact with the heart wall for sensing a target site in heart 12. For ablation, the physician may similarly place a distal end of an ablation catheter in contact with a target site for ablating tissue.

System 10 includes one or more electrode patches 38 positioned for skin contact on patient 23 to establish location reference for location pad 25 as well as impedance-based tracking of electrodes 26. For impedance-based tracking, electrical current is directed to electrodes 26 and sensed at electrode skin patches 38 so that the location of each electrode can be triangulated via the electrode patches 38. Details of the impedance-based location tracking technology are described in U.S. Pat. Nos. 7,536,218; 7,756,576; 7,848,787; 7,869,865; and 8,456,182.

A recorder 11 records and displays electrograms 21 captured with body surface ECG electrodes 18 and intracardiac electrograms (IEGM) captured with electrodes 26 of catheter 14. Recorder 11 may include pacing capability for pacing the heart rhythm and/or may be electrically connected to a standalone pacer.

System 10 may include an ablation energy generator 50 that is adapted to conduct ablative energy to one or more of electrodes at a distal tip of a catheter configured for ablating. Energy produced by ablation energy generator 50 may include, but is not limited to, radiofrequency (RF) energy or pulsed-field ablation (PFA) energy, including monopolar or bipolar high-voltage DC pulses as may be used to effect irreversible electroporation (IRE), or combinations thereof.

Patient interface unit (PIU) 30 is an interface configured to establish electrical communication between catheters, other electrophysiological equipment, power supply and a workstation 55 for controlling operation of system 10. Electrophysiological equipment of system 10 may include for example, multiple catheters, location pad 25, body surface ECG electrodes 18, electrode patches 38, ablation energy generator 50, and recorder 11. Optionally and preferably, PIU 30 additionally includes processing capability for implementing real-time computations of location of the catheters and for performing ECG calculations.

Workstation 55 includes memory, processor unit with memory or storage with appropriate operating software stored therein, and user interface capability. Workstation 55 may provide multiple functions, optionally including (1) modeling the endocardial anatomy in three-dimensions (3D) and rendering the model or anatomical map 20 for display on a display device 27, (2) displaying on display device 27 activation sequences (or other data) compiled from recorded electrograms 21 in representative visual indicia or imagery superimposed on the rendered anatomical map 20, (3) displaying real-time location and orientation of multiple catheters within the heart chamber, and (4) displaying on display device 27 sites of interest such as places where ablation energy has been applied. One commercial product embodying elements of the system 10 is available as the CARTO™ 3 System, available from Biosense Webster, Inc., 31A Technology Drive, Irvine, CA 92618.

In some examples, an irrigation module is provided for delivering irrigation fluid, such as saline solution, to the location of treatment. The irrigation module may comprise a pump and an associated fluid tank.

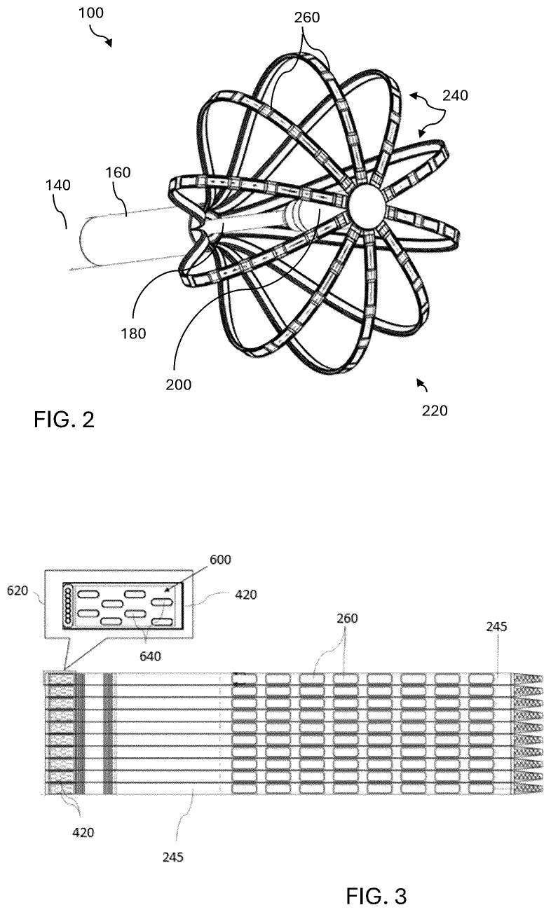

Reference is now made to FIG. 2, which is schematic view of a catheter assembly 100 constructed and operative in accordance with examples of the present disclosure. The catheter assembly 100 include a tubular shaft 140 having a distal end, a coupler (also referred to as spline retention hub) 160 connected to the distal end 140 of the tubular shaft and a pusher 180 including a distal portion 200. The pusher 180 may be configured to be advanced and retracted through the tubular shaft, for example, using a manipulator or handle (not shown). The pusher 180 may be disposed coaxially within the spline retention hub and configured to be translated longitudinally relative thereto. The catheter assembly 100 also includes an expandable assembly 220 (also referred to generally as expandable end effector) comprising a plurality of flexible splines 240 (only one label for the sake of simplicity). Each flexible spline 240 includes a flexible circuit strip (PCB) including multiple electrodes 260 disposed thereon (only some labeled for the sake of simplicity) and a corresponding elongated resilient support element extending along a given length of the flexible polymer circuit strip. The flexible splines 240 may be coupled at one end to the coupler 160 and at the other end to the distal end 200 of the pusher 180 so that actuation of the pusher 180 may selectively expand or retract the expandable assembly 220. Typically, the catheter assembly 100 may be inserted into a delivery sheath (not shown in FIG. 2) so as to arrive at the desired location in heart 12. Thereafter, the expandable assembly 220 may be expanded when protruding out of the delivery sheath by actuation of the pusher 180.

The flexible circuit strip may have any suitable dimensions. For example, the length of the flexible circuit strip may be in the range of 10 mm to 60 mm, e.g., 30 mm, the width of the flexible circuit strip may be in the range of 0.25 mm to 3 mm, e.g., 0.72 mm, the thickness of the flexible circuit strips may be in the range of 0.005 mm to 0.14 mm.

In some examples, electrodes 260 extend to occupy a majority of the width of the flexible circuit strips by which they are hosted. Where, in some examples, electrode width is 0.25-3 mm, and/or electrode length is 0.25-3 mm.

The elongated resilient support elements may provide a shape of the expandable assembly 220 in the expanded form of the expandable assembly 220. The elongated resilient support elements may include any suitable material, for example, but not limited to, Nitinol and/or shape-set Nitinol and/or Polyetherimide (PEI). As explained in further details herein, the present disclosure may provide that the respective elongated resilient support elements may extend in a hollow channel of the respective flexible circuit strips.

Electrodes 260 positioned on splines 240 of basket assembly 220 can be configured to determine the location of basket assembly 220 and/or to measure a physiological property such as local surface electrical potentials at respective locations on cardiac tissue. Additionally, or alternatively, the electrodes can also be used to deliver ablation energy RF and/or IRE to cardiac tissue. Examples of materials ideally suited for forming electrodes 220 include gold, platinum and palladium and their respective alloys.

In other examples, the catheter assembly may include a tubular shaft having a distal end, a coupler connected to the distal end of the tubular shaft. The catheter assembly may also include an expandable assembly 220 (also referred to generally as expandable end effector) comprising a plurality of flexible splines (only one label for the sake of simplicity). Each flexible spline may include a flexible circuit strip (PCB) including multiple electrodes disposed thereon and a corresponding elongated resilient support element extending along a given length of the flexible polymer circuit strip.

The flexible splines may be coupled at both ends to the coupler (also referred to as spline retention hub) and may be configured to expand radially into a basket shape when unconstrained. Typically, the catheter assembly may be inserted into a delivery sheath so as to arrive at the desired location in heart. Thereafter, the expandable assembly may expand when protruding out of the delivery sheath i.e. only by being advanced out of the delivery sheath. In other words, in these other examples, the expandable basket assembly may be configured to transition from the collapsed form to the expanded form solely by release of radial constraints on the flexible splines. The flexible splines may also be configured to retract when being pulled into the delivery sheath.

The catheter assembly may further include a contact force sensor to determine contact force of the splines against cardiac tissues. Details of the contact force sensor are shown and described in U.S. Patent Application Publication No. US20210077180A1 published Mar. 18, 2021, which disclosure is incorporated by reference herein.

Reference is now made to FIG. 3, which is a schematic view of flexible polymer circuit strips for use e.g. in the catheter assembly 100 of FIG. 2. The flexible polymer circuit strips 245 may be formed from a polymer, such as polyimide. Circuit strips 245 may be connected to each other by polyimide, or assembled as individual pieces that are held in proper alignment and secured to coupler 160. Respective first ends 420 of the respective flexible polymer circuit strips 245 include an electrical connection array 600. An inset 62 shows that the electrical connection array 600 includes electrical contacts 640 thereon (only some labeled for the sake of simplicity). The electrical contacts 640 are connected via traces (not shown) on one side of top layer of the flexible polymer circuit strips 245 to respective ones of the electrodes 260 disposed on the other side of the top layer of the flexible polymer circuit strips. Away from the region of the first ends 420, the flexible polymer circuit strips 245 are separate from each other to allow the flexible polymer circuit strips 245 to form the expandable assembly 220 (FIG. 2) when connected to the catheter assembly 100. Wires (not shown) may connect the electrodes 260 to control circuitry (not shown) via the electrical contacts 640. The wires may be disposed in lumens (not illustrated) of the elongated deflectable element.

In some examples, one or more pad dimensions are smaller than electrode dimensions. Where the surface area the pad presents for contact (e.g., by a measurement probe or joining tool) is smaller than that presented by the electrodes. For example, a pad upper surface area being 1-20% of that of the electrodes. For example, a pad width 194 being 0.05-0.5 mm, and/or a pad length 0.1-1 mm.

A surface area 620 of the flexible circuit strips on which pads 420 are disposed may be small and/or the density of pads on the area may be high. For example, the area having a width of 0.25-3 mm and a length of 0.5-6 mm and/or the proportion of the surface area occupied by pads is 20-50%.

As explained in more details below, the flexible polymer circuit strip is generally formed from multiple insulating layers including a top layer (also referred to as first layer), a middle layer (also referred to as second layer) and a bottom layer (also referred to as third layer). A conductive trace layer may be positioned between the middle insulating layer and the top layer of the flexible polymer circuit strip 245. The conductive trace layer may include circuit traces which connect the electrodes 260 to the connection array 600. The top layer includes the electrodes 260 and optionally one or more contact pads. The circuit traces on the middle layer may connect each electrode 260 to a corresponding electrical contact 640 using vias (not shown). The circuit traces for the electrodes 260 may be within the range of about 0.005 mm to 0.1 mm wide (e.g., 0.025 mm) with a spacing in the range of about 0.005 mm to 0.1 mm (e.g., 0.025 mm). The thickness of the traces may be in the range of about 0.005 mm to 0.100 mm (e.g., 0.010 mm). In some embodiments, the circuit traces may have the same width and spacing.

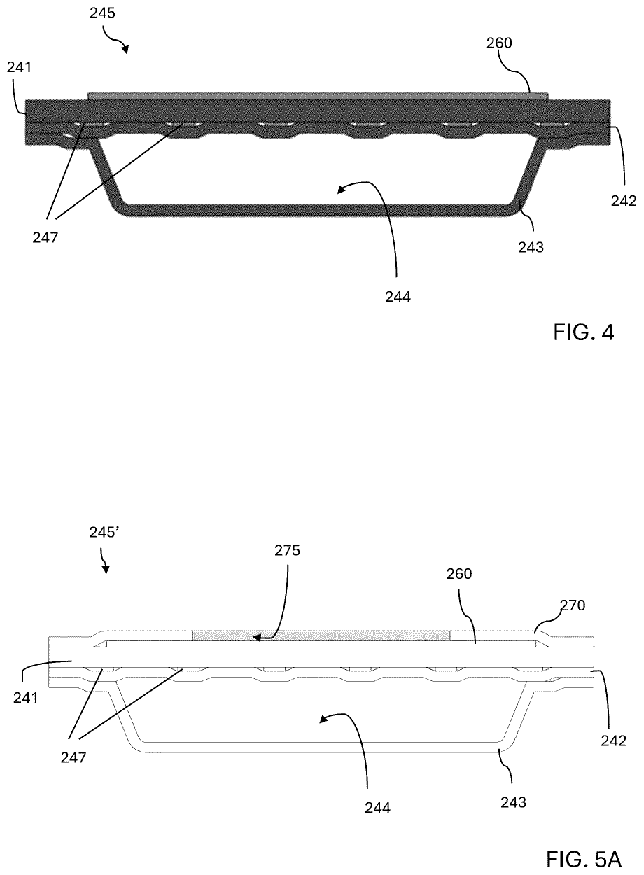

FIG. 4 illustrates a cross section view of a flexible circuit strip 245 in accordance with examples of the present disclosure. Flexible circuit strip 245 may include a (top) first insulating layer 241, a second (middle) insulating layer 242 and a third (bottom) insulating layer 243. The first, second and third insulating layers 241, 242, 243 may be formed from a polymer, such as polyimide. The first, second and third insulating layers 241, 242, 243 may have elongated shapes so as to form a circuit strip. The first, second and third insulating layers 241, 242, 243 may have corresponding lateral and longitudinal dimensions so that they can be properly aligned and registered to each other e.g. in a lamination process. A thickness of the first insulating layer 241 may be larger than one of the second and third insulating layers 242, 243.

A proximal end of the first insulating layer 241 may include an electrical connection array (also referred to as contact array) as described in relation to FIG. 3. The electrical connection array may be positioned at a proximal end of the outer surface of the first insulating layer 241. Electrodes 260 may be positioned along a length of an outer surface of the first insulating layer 241. The electrodes 260 may be disposed away from the proximal end of the insulating layer 241. The electrodes may be formed of a first conductive material such as gold.

The flexible circuit strip 245 may further include a conductive trace layer positioned between the first insulating layer 241 and the second insulating layer 242. The conductive trace layer may include longitudinal circuit traces 247 for carrying electrical signals. The longitudinal circuit traces 247 may be configured to carry electrical signals between the electrodes 260 and the electrical connection array. The electrodes 260 may be connected to the circuit traces 247 using vias extending through the first and second insulating layers 241, 242. The conductive circuit traces 247 may be formed of the same first conductive material as the electrodes. In some embodiments, the conductive circuit traces may be formed of a second conductive material different from the first conductive material.

The flexible circuit strip 245 may further include a longitudinal hollow channel 244 formed between the second and third insulating layers 242, 243 (or in some other examples between the first and second insulating layers). The longitudinal hollow channel 244 may be configured to receive an elongated support element (not shown) extending substantially along the full length of the flexible circuit strip. The elongated support element may be formed of resilient material and may include, for example, Nitinol and/or shape-set Nitinol and/or Polyetherimide (PEI). The elongated support element may be arranged in the longitudinal hollow channel to have play, allowing for longitudinal adjustment therein. The elongated support element may be adhered to the longitudinal hollow channel using epoxy so as to provide a limited adhesion allowing for residual longitudinal play. In some examples, the elongated support element may be adhered so as to be fixed within the longitudinal channel. The longitudinal hollow channel may have a constant cross section geometry. A cross sectional shape of the longitudinal hollow channel may be trapezoidal, rectangular, circular or elliptical. In some examples, the longitudinal hollow channel may have a variable cross section along the length of the flexible circuit strip and may be configured to accommodate an elongated support element with a varying diameter. In some examples, the longitudinal hollow channel may comprise reinforcement features to enhance structural integrity of the flexible circuit strip.

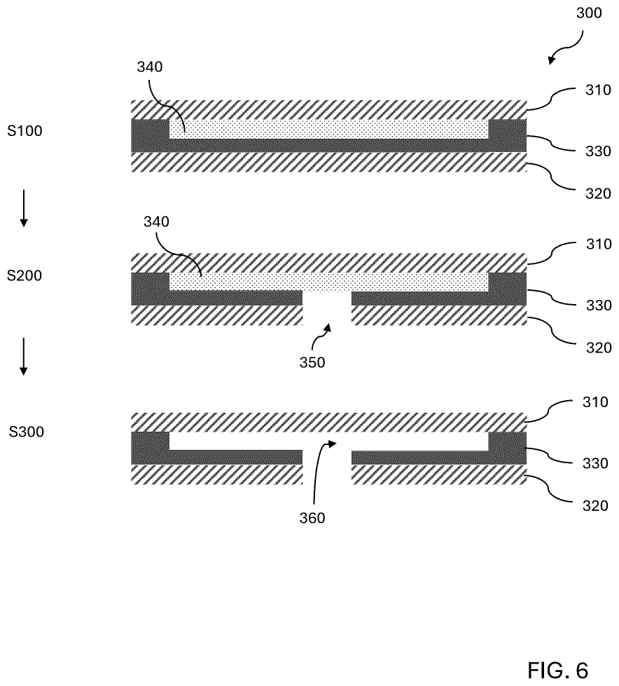

As explained in more details with reference to FIG. 6, the longitudinal hollow channel 244 may be created by etching away a conductive channel layer clad to a surface of the second insulating layer facing the third insulating layer. The conductive channel layer may be formed of a third conductive material (e.g. copper) different from the first and/or second conductive material. This may allow to etch the conductive layer clad while preserving the electrodes and/or circuit traces.

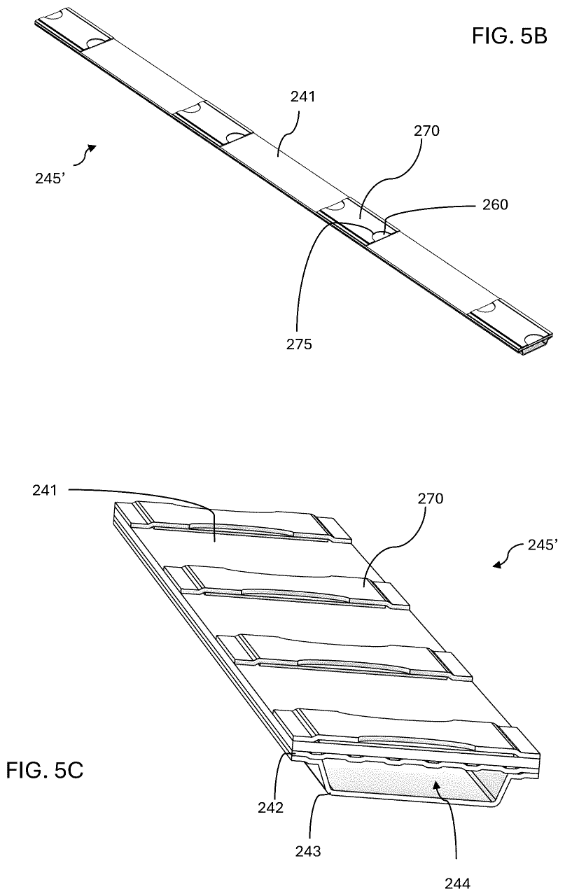

FIGS. 5A-5C illustrate views of a flexible circuit strip 245′ in accordance with examples of the present disclosure. In FIGS. 5A-65, the same reference numbers are used to represent elements that are similar to those in the previously described FIG. 4. For the sake of brevity, the detailed description of these elements is not repeated here. Only the additions or differences are elaborated upon. As illustrated in FIGS. 5A-5C, the flexible circuit strip 245′ additionally includes a plurality of insulating covers 270 respectively covering the plurality of electrodes 260. The insulating covers 270 may include one or more apertures 275. The insulating covers 270 may be positioned over the electrodes 260 so as to expose selected portions of the electrodes 260. The one or more apertures 275 may have a semi-circular shape so as to expose a semi-circular portion of the electrode. In some examples, the selected portions of the electrodes may be positioned at lateral edges of the electrodes 260.

The flexible circuit strips 245, 245′ described with reference to FIGS. 4 and 5A-5C may be suitable for integration into the end effector and catheter assemblies detailed above with reference to FIG. 2. In particular, the flexible splines 240 described herein above may include the circuit strips 245, 245′ and an elongated support element may be disposed in the longitudinal hollow channel 244 of said circuit strips 245, 245′.

FIG. 6 illustrates steps of a method for fabricating a flexible circuit strip in accordance with examples of the present disclosure. In a first step S100, an elongated multilayer laminate 300 may be provided. The multilayer laminate 300 may comprise an insulating layer 310 formed of a flexible polymer such as polyimide, a conductive channel layer 340 clad onto the insulating layer 310 formed of a conductive material such as copper, an adhesive layer 330 formed of an adhesive such as epoxy and another insulating layer 320 formed of polymer such as polyimide. In a second step S200, the multilayer laminate 300 may be drilled to form an opening 350 through the other polymer layer 320 and the adhesive layer 330 so as to expose the conductive channel layer 340. This step may be performed using precision drilling techniques such as laser drilling or mechanical drilling to ensure accuracy and consistency. In a third step S300, the conductive channel layer 340 may be etched so as to form a longitudinal hollow channel in the elongated multilayer laminate 300. The etching process may involve chemical etching using an etchant solution that selectively removes the conductive material of the conductive channel (e.g. copper) while preserving other conductive materials optionally present on the laminate (e.g. gold traces and/or electrodes), or plasma etching for more precise control.

The method described herein may be used to form the longitudinal hollow channel in the flexible circuit strips as described herein above. The insulating layers 310, 320 may correspond to the middle insulating layer and bottom insulating layer as described previously. The resulting hollow channel may be configured to accommodate an elongated support element, such as a nitinol wire.

### EXAMPLES

Following is a non-exclusive list of some exemplary examples of the disclosure. The present disclosure also includes examples which include fewer than all the features in an example and examples using features from multiple examples, even if not listed below.

### Example 1

A flexible circuit strip (245) for use in a catheter assembly (100), comprising:
(a) A first insulating layer (241), a second insulating layer (242) and a third insulating layer (243), each having an elongated shape and bonded together such that the second insulating layer is between the first and third insulating layers;
(b) a conductive trace layer positioned between the first and second insulating layers, the conductive trace layer including longitudinal circuit traces (247) for carrying electrical signals;
(c) electrodes (260) positioned along a length of an outer surface of the first insulating layer and electrically connected to the circuit traces;
(d) a longitudinal hollow channel (244) formed between any two of the first, second and third layers (241, 242, 243), the longitudinal hollow channel (244) being configured to receive an elongated support element.

### Example 2

The flexible circuit strip (245) of Example 1, further comprising a contact array (600) positioned at a proximal end of the outer surface of the first insulating layer (241), wherein the electrodes (260) are connected to the contact array (600) using the circuit traces (247).

### Example 3

The flexible circuit strip (245) of Example 2, wherein the connection between the circuit traces (247) and the electrodes (260) is established through vias extending through the first insulating layer (241).

### Example 4

The flexible circuit strip (245) of any of the preceding Examples, wherein each electrode (260) is covered by an insulating cover (270) including one or more apertures (275), the insulating cover (270) being positioned over the electrode (260) to expose selected portions of the electrodes (260).

### Example 5

The flexible circuit strip (245) of any of the preceding Examples, wherein the electrodes (260) and the circuit traces (247) comprise gold.

### Example 6

The flexible circuit strip (245) of any of the preceding Examples, wherein the first, second, and third insulating layers (241, 242, 243) comprises polyimide.

### Example 7

The flexible circuit strip (245) of any of the preceding Examples, wherein the conductive channel layer comprises gold.

### Example 8

The flexible circuit strip (245) of any of the preceding Examples, wherein the first insulating layer (241) is thicker than the second and third insulating layers (242, 243).

### Example 9

An expandable end effector for use in a catheter assembly (100), the expandable end effector (220) comprising:
(a) a spline retention hub (160);
(b) an expandable basket assembly (220) coupled to the spline retention hub (160), the expandable basket assembly (220) comprising one or more flexible spline (240) coupled to the spline retention hub (160) and being configured to bow radially outward from a collapsed form to an expanded form, each flexible spline (240) including:
   i. a flexible circuit strip (245) according to any of the preceding Examples;
   ii. an elongated support element disposed in the longitudinal hollow channel (244).

### Example 10

The expandable end effector of Example 9, wherein the elongated support member is arranged to have play within the longitudinal hollow channel (244), allowing for longitudinal adjustment therein.

### Example 11

The expandable end effector of Example 9, wherein the elongated support member is fixed within the longitudinal hollow channel (244).

### Example 12

The expandable end effector of any of Examples 9 to 11, wherein the expandable basket assembly (220) comprises a first flexible spline having a proximal end coupled to the spline retention hub (160) and a distal end of a first flexible spline coupled to a distal end of a second flexible spline.

### Example 13

The expandable end effector of any of Example 9 to 11, further comprising a pusher (180) disposed coaxially within the spline retention hub (160) and configured to be translated relative thereto along a hub longitudinal axis; each of the one or more flexible spline (240) having a proximal end coupled to the spline retention hub (160) and a distal end coupled to a distal end of the pusher (180), the one or more flexible spline (240) being configured to bow radially outward when the pusher (180) is retracted relative to the spline retention hub (160), thereby expanding the expandable basket assembly (220) from a collapsed form to an expanded form.

### Example 14

The expandable end effector of any of Example 9 to 11, wherein the one or more flexible spline (240) is configured to bow radially outward upon release of radial constraint.

### Example 15

The expandable end effector of Example 14, wherein the one or more flexible spline (240) comprises a proximal end and a distal end both coupled to the spline retention hub (160).

### Example 16

The expandable end effector of any of Examples 9 to 15, comprising a plurality of flexible splines (240) forming a basket in the expanded form.

### Example 17

The expandable end effector of any of Examples 9 to 16, wherein the elongated support element is fitted to the longitudinal hollow channel (244) and secured loosely using an adhesive so as to have longitudinal play.

### Example 18

The expandable end effector of any of Examples 9 to 17, wherein the elongated support element comprises shape-set nitinol.

### Example 19

A catheter assembly (100) comprising:
a tubular shaft (140) extending along a longitudinal axis extending from a proximal portion of the tubular shaft (140) to a distal portion of the tubular shaft (140); and
an expandable end effector according to any of Examples 9 to 18.

### Example 20

A method of manufacturing a flexible circuit strip according to any of examples 1 to 8, the method comprising:
(a) providing (S100) an elongated multilayer laminate (300), wherein the multilayer laminate comprises an insulating layer (310) comprising a flexible polymer, a conductive channel layer (340) clad onto the insulating layer (310) and comprising a conductive material, an adhesive layer (330) and another insulating layer (320) comprising said flexible polymer;
(b) drilling (S200) the multilayer laminate 300) to form an opening (350) through said another polymer layer (320) and the adhesive layer (330) to expose the conductive channel layer (340);
(c) etching the conductive channel layer (340) to form a longitudinal hollow channel (360) in the elongated multilayer laminate (300).

Those skilled in the art to which the present disclosure pertains, can appreciate that while the present disclosure has been described in terms of preferred examples, the concept upon which this disclosure is based may readily be utilized as a basis for the designing of other structures, systems and processes for carrying out the several purposes of the present disclosure.

Also, it is to be understood that the phraseology and terminology employed herein are for the purpose of description and should not be regarded as limiting. It should be noted that the words “comprising”, “including” and “having” as used throughout the appended claims are to be interpreted to mean “including but not limited to”. The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases, and disjunctively present in other cases. The term “each” may not be exclusively understood as referring to each and every, and when technically relevant may also refer to “at least some”.

All patents and patent applications mentioned in this specification are herein incorporated in their entirety by reference into the specification, to the same extent as if each individual patent or patent application was specifically and individually indicated to be incorporated herein by reference. In addition, citation or identification of any reference in this application shall not be construed as an admission that such reference is available as prior art to the present disclosure.

It is important, therefore, that the scope of the present disclosure is not construed as being limited by the illustrative examples set forth herein. Other variations are possible within the scope of the present disclosure as defined in the appended claims. Other combinations and sub-combinations of features, functions, elements and/or properties may be claimed through amendment of the present claims or presentation of new claims in this or a related application. Such amended or new claims, whether they are directed to different combinations or directed to the same combinations, whether different, broader, narrower or equal in scope to the original claims, are also regarded as included within the subject matter of the present description.

What is claimed is:

1. A flexible circuit strip for use in a catheter assembly, comprising:
(a) a first insulating layer, a second insulating layer and a third insulating layer, each having an elongated shape and bonded together such that the second insulating layer is between the first and third insulating layers;
(b) a conductive trace layer positioned between the first and second insulating layers, the conductive trace layer including longitudinal circuit traces for carrying electrical signals;
(c) electrodes positioned along a length of an outer surface of the first insulating layer and electrically connected to the circuit traces;
(d) a longitudinal hollow channel formed between any two of the first, second and third layers, the longitudinal hollow channel being configured to receive an elongated support element.

2. The flexible circuit strip of claim 1, further comprising a contact array positioned at a proximal end of the outer surface of the first insulating layer, wherein the electrodes are connected to the contact array using the circuit traces.

3. The flexible circuit strip of claim 2, wherein the connection between the circuit traces and the electrodes is established through vias extending through the first insulating layer.

4. The flexible circuit strip of claim 1, wherein each electrode is covered by an insulating cover including one or more apertures, the insulating cover being positioned over the electrode to expose selected portions of the electrodes.

5. The flexible circuit strip of claim 1, wherein the electrodes and the circuit traces comprise gold.

6. The flexible circuit strip of claim 1, wherein the first, second, and third insulating layers comprises polyimide.

7. The flexible circuit strip of claim 1, wherein the conductive trace layer comprises gold.

8. The flexible circuit strip of claim 1 wherein the first insulating layer is thicker than the second and third insulating layers.

9. An expandable end effector for use in a catheter assembly, the expandable end effector comprising:
(a) a spline retention hub;
(b) an expandable basket assembly coupled to the spline retention hub, the expandable basket assembly comprising one or more flexible splines coupled to the spline retention hub and being configured to bow radially outward from a collapsed form to an expanded form, each flexible spline including:
i. a flexible circuit strip comprising:
a first insulating layer, a second insulating layer and a third insulating layer, each having an elongated shape and bonded together such that the second insulating layer is between the first and third insulating layers;
a conductive trace layer positioned between the first and second insulating layers, the conductive trace layer including longitudinal circuit traces for carrying electrical signals;
electrodes positioned along a length of an outer surface of the first insulating layer and electrically connected to the circuit traces;
a longitudinal hollow channel formed between any two of the first, second and third insulating layers, the longitudinal hollow channel being configured to receive an elongated support element;
ii. an elongated support element disposed in the longitudinal hollow channel.

10. The expandable end effector of claim 9, wherein the elongated support element is arranged to have play within the longitudinal hollow channel, allowing for longitudinal adjustment therein.

11. The expandable end effector of claim 9, wherein the elongated support element is fixed within the longitudinal hollow channel.

12. The expandable end effector of claim 9, wherein the expandable basket assembly comprises a first flexible spline having a proximal end coupled to the spline retention hub and a distal end of a first flexible spline coupled to a distal end of a second flexible spline.

13. The expandable end effector of claim 9, further comprising a pusher disposed coaxially within the spline retention hub and configured to be translated relative thereto along a hub longitudinal axis; each of the one or more flexible spline having a proximal end coupled to the spline retention hub and a distal end coupled to a distal end of the pusher, the one or more flexible spline being configured to bow radially outward when the pusher is retracted relative to the spline retention hub, thereby expanding the expandable basket assembly from a collapsed form to an expanded form.

14. The expandable end effector of claim 9, wherein the one or more flexible spline is configured to bow radially outward upon release of radial constraint.

15. The expandable end effector of claim 14, wherein the one or more flexible spline comprises a proximal end and a distal end both coupled to the spline retention hub.

16. The expandable end effector of claim 9, comprising a plurality of flexible splines forming a basket in the expanded form.

17. The expandable end effector of claim 9, wherein the elongated support element is fitted to the longitudinal hollow channel and secured loosely using an adhesive so as to have longitudinal play.

18. The expandable end effector of claim 9, wherein the elongated support element comprises shape-set nitinol.

19. A catheter assembly comprising:
a tubular shaft extending along a longitudinal axis extending from a proximal portion of the tubular shaft to a distal portion of the tubular shaft; and
an expandable end effector comprising:
(a) a spline retention hub coupled to the distal portion of the tubular shaft;
(b) an expandable basket assembly coupled to the spline retention hub, the expandable basket assembly comprising one or more flexible spline coupled to the spline retention hub and being configured to bow radially outward from a collapsed form to an expanded form, each flexible spline including:
i. a flexible circuit strip comprising:
a first insulating layer, a second insulating layer and a third insulating layer, each having an elongated shape and bonded together such that the second insulating layer is between the first and third insulating layers;
a conductive trace layer positioned between the first and second insulating layers, the conductive trace layer including longitudinal circuit traces for carrying electrical signals;
electrodes positioned along a length of an outer surface of the first insulating layer and electrically connected to the circuit traces;
a longitudinal hollow channel formed between any two of the first, second and third insulating layers, the longitudinal hollow channel being configured to receive an elongated support element;
ii. an elongated support element disposed in the longitudinal hollow channel.

20. A method of manufacturing a flexible circuit strip, comprising:
(a) providing an elongated multilayer laminate, wherein the multilayer laminate comprises an insulating layer comprising a flexible polymer, a conductive channel layer clad onto the insulating layer and comprising a conductive material, an adhesive layer and another insulating layer comprising said flexible polymer;
(b) drilling the multilayer laminate to form an opening through said another polymer layer and the adhesive layer to expose the conductive channel layer;
(c) etching the conductive channel layer to form a longitudinal hollow channel in the elongated multilayer laminate.
